(19) United States
(12) Patent Application Publication
(10) Pub. No.: US 2026/0309621 A1
(43) Pub. Date: Oct. 8, 2026

(54) HIGH THROUGHPUT OPTICAL ASSAY FOR SCREENING HEMOGLOBIN OXYGEN AFFINITY MODIFYING DRUG

(71) Applicant: CASE WESTERN RESERVE UNIVERSITY, Cleveland, OH (US)

(72) Inventors: Umut Gurkan, Cleveland, OH (US); Zoe Sekyonda, Cleveland, OH (US); Ozan Akkus, Cleveland, OH (US); Ran An, Cleveland, OH (US)

(21) Appl. No.: 19/481,630

(22) PCT Filed: May 6, 2024
(86) PCT No.: PCT/US2024/027974

§ 371 Date: Nov. 5, 2025

Related U.S. Application Data
(60) Provisional application No. 63/464,246, filed on May 5, 2023.

(51) Int. Cl. G01N 21/31; G01N 21/25; G01N 33/72

(52) U.S. Cl. CPC G01N 21/31; G01N 21/253; G01N 33/721; G01N 2500/04

(57) ABSTRACT

A high throughput assay for screening hemoglobin oxygen affinity modifying agents, the assay includes a well plate configured to receive samples of hemoglobin, red blood cells, or a blood sample that has been deoxygenated and at least one oxygen affinity modifying agent; a UV-VIS spectroscopic microplate reader that is configured to determine an optical signature of hemoglobin, red blood cells, or blood in each well of the well plate; and a processor configured for comparing the determined optical signature to a control optical signature in each of the wells wherein differences between the determined optical signature and the control optical signature is indicative of the agents' effect on hemoglobin oxygen affinity.

### RELATED APPLICATION

This application claims priority from U.S. Provisional Application No. 63/464,246, filed May 5, 2023, the subject matter of which is incorporated herein by reference in its entirety.

### GOVERNMENT FUNDING

This invention was made with government support under HL133574 and HL152643 awarded by the National Institute of Health (NIH). The government has certain rights in the invention.

### BACKGROUND

Sickle Cell Disease (SCD) affects millions of people worldwide and is associated with vaso-occlusion, ischemia, inflammation, significant morbidity, and early mortality. In SCD, abnormal polymerization of deoxygenated sickle hemoglobin (HbS) deforms red blood cells (RBCs), changing their biochemical, affinity for oxygen and viscoelastic properties, resulting in harmful inflammation, thrombophilia, and vasculopathy. Sickle RBCs are non-deformable and adhesive in the microcirculation, particularly in areas of the body where the oxygen tension is relatively low (hypoxic), such as the kidney or spleen. In the developed world, the iterative development of management strategies has increased the survival rate and quality of life for people with SCD. However, the burden of cumulative morbidity and increased mortality from chronic end-organ damage remains significant.

In recent years, the potential of improving sickle cell therapy by boosting oxygen affinity has grown. Increased oxygen affinity may aid increase sickling delay duration by lowering polymerization. The US Food and Drug Administration approved voxelotor (previously known as GBT440), an oxygen affinity modifying drug, for the treatment of SCD in 2019, and several other drugs are in advanced clinical trials such as Etavopivate, a Pyruvate Kinase Activator (PKA), that targets intercellular organic molecules including 2, 3 DPG that affect oxygen affinity and polymerization. However, despite the widely recognized potential of these new oxygen-modifying drugs, there is still a lack of a comprehensive tool that can determine the efficacy of those drugs. Thus, there is a significate need for an assay that can be used to test the effectiveness and screen out oxygen-modifying treatment.

### SUMMARY

Embodiments described herein relate to a high throughput assay for screening hemoglobin (Hb) oxygen affinity modifying agents. The assay takes advantage of differing Hb oxygenation and deoxygenation optical absorption spectra. It was found that under specific deoxygenation and pH levels, absorption spectra of sickle cell disease (SCD) Hb (e.g., HbS) samples had a larger bathochromic and hypochromic shift magnitude under deoxygenation at all pH levels compared to normal healthy blood containing HbA, where the higher the magnitude of the shift, the lower the oxygen affinity of HbS. We found that treated SCD samples with the oxygen modifying agents had reduced peak wavelength shift compared to untreated SCD samples, which means that the oxygen modifying molecules were effectively increasing the oxygen affinity. An increase in the concentrations of the oxygen modifying agents further induced a decrease in the peak wavelength shift. Thus, optical absorption spectra of the SCD sample can be indicative of an oxygen affinity modifying agent's effect on hemoglobin oxygen affinity and particularly hemoglobin oxygen affinity of HbS of SCD samples.

In some embodiments, the assay can include a well plate configured to receive a plurality of hemoglobin, red blood cells, or blood samples that have been deoxygenated and at least one oxygen affinity modifying agent, a UV-VIS spectroscopic microplate reader that is configured to determine an optical signature of hemoglobin, red blood cells, or blood in each well, and a processor configured for comparing the determined optical signature to a control optical signature in each well. Differences between the determined optical signature and the control optical signature is indicative of the agents' effect on hemoglobin oxygen affinity.

In some embodiments of the assay, the processor is configured to determine differences of absorption spectra of oxygenated and/or deoxygenated hemoglobin, red blood, and/or blood obtained from the subject administered an oxygen affinity modifying agent and compare the determined absorption spectra differences to a control value.

In some embodiments of the assay, the processor is configured to determine differences of absorption spectra by comparing a first optical absorption spectrum of oxygenated and/or deoxygenated hemoglobin, red blood cells, or blood with a second optical absorption spectrum of deoxygenated hemoglobin, red blood cells, or blood after administrating of the oxygen affinity modifying agent.

In other embodiments of the assay, the processor is configured to determine differences in at least one of a bathochromic shift and/or hypochromic shift in peak wavelength from the first absorption spectra to the second absorption spectra.

In some embodiments of the assay, the magnitude of bathochromic shift in peak wavelength is indicative of at least one hemoglobin oxygen affinity or rate of hemoglobin deoxygenation.

In some embodiments of the assay, an increase in magnitude of biochromatic shift and/or hypochromic shift in peak wavelength compared to a control is indicative of decreased hemoglobin oxygen affinity or increased hemoglobin deoxidation.

In some embodiments of the assay, processor is configured to determine differences of area under a curve of and/or full width half maximum of peak wavelengths of the first absorption spectra and the second absorption spectra are indicative oxygen affinity modifying effect of the agent.

In some embodiments of the assay, the absorption spectra of the oxygenated and chemically deoxygenated hemoglobin are measured at the same pH from about 6.5 to about 7.0, preferably about 6.8 to less than about 7.0, or more preferably, about 6.9.

In some embodiments of the assay, the hemoglobin, red blood cells, and/or blood is chemically deoxygenated by mixing the hemoglobin, red blood cells, and/or blood with an amount of chemical deoxygenant, such as sodium metabisulfite, effective to deplete oxygen from the hemoglobin.

Other embodiments described herein relate to a high throughput method for screening oxygen affinity modifying agents. The method includes mixing hemoglobin, red blood cells, and/or whole blood samples with a buffer solution. The mixed hemoglobin, red blood, and/or blood samples and buffer solution is deoxygenated. The mixed hemoglobin, red blood, and/or blood samples and buffer solution is added to wells of a well plate. At least one oxygen affinity modifying agent from a library of agents is added to the wells. The absorption spectra of deoxygenated hemoglobin, red blood, and/or blood in the wells is determined. The determined absorption spectra is compared to a control absorption spectra of oxygenated and/or deoxygenated hemoglobin, red blood, and/or blood mixed with a similar buffer solution but without the oxygen affinity modifying agent. The absorption spectra differences are indicative of the agent's effect on modulating hemoglobin oxygen affinity of the hemoglobin, red blood, and/or blood.

In some embodiments of the method, the differences of the absorption spectra include at least one of a bathochromic shift and/or hypochromic shift in peak wavelength from the first absorption spectrum to the second absorption spectrum.

In some embodiments of the method, the magnitude of bathochromic shift in peak wavelength is indicative of at least one hemoglobin oxygen affinity or rate of hemoglobin deoxygenation.

In some embodiments of the method, an increase in magnitude of bathochromic shifty and/or hypochromic shift in peak wavelength is indicative of decreased hemoglobin oxygen affinity or increased hemoglobin deoxidation.

In some embodiments of the method, the hemoglobin, red blood cells, and/or blood is chemically deoxygenated by mixing the hemoglobin, red blood cells, and/or blood with an amount of chemical deoxygenant, such as metabisulfite, effective to deplete oxygen from the hemoglobin.

### BRIEF DESCRIPTION OF THE DRAWINGS

FIG. 1 illustrates a block diagram showing an assay in accordance with an embodiment.

FIG. 2 illustrates a flow chart showing a method in accordance with an embodiment.

FIGS. 3(A-C) illustrate varying concentration of voxelotor tested using 96 well plates optical assay. (A) Representative 96 well plate, showing the arrangement of a single sample analyzed. The plate included oxygenated HbAA (Oxy HbAA), oxygenated HbSS (Oxy HbSS), Deoxygenated HbAA (deoxy HbAA), Deoxygenated HbSS (deoxy HbSS), and voxelotor treated samples in the concentration range of 20 μM-260 μM (HbSS+VoX). (B) Insert from the 12 columns of the 96 well plate, showing the Soret band peak wavelength shift in the wavelength range of 380-450 nm. The right-most plot shows the zoomed-in region in the wavelength range of 390-440 nm for the HbSS sample (C) Significate differences (Mann-Whitney) were observed between deoxy HbSS and deoxy HbSS+20 μM Vox, HbSS+65 μM Vox, HbSS+130 μM Vox and HbSS+260 μM Vox with P=0.001, P=0.67, P=0.03 respectively.

FIGS. 4(A-B) illustrate increasing the concentration of voxelotor induces a decrease in the peak wavelength of SCD samples (A) Insert from the 12 columns of the 96 well plate, showing the Soret band peak wavelength shift in the wavelength range of 380-450 nm. (C) Significate differences (Mann-Whitney) were observed between deoxy HbSS and deoxy HbSS+63 μM Vox, HbSS+118 μM Vox, HbSS+211 μM Vox and HbSS+421 μM Vox. Those results suggest that increasing the concentration of voxelotor increases the oxygen affinity of HbSS as this is depicted by the reduction in the peak wavelength shift to that of HbAA.

FIGS. 5(A-B) illustrate varying concentration of PKR activator tested using 96 well plates optical assay. (A) Insert from the 12 columns of the 96 well plate, showing the Soret band peak wavelength shift in the wavelength range of 380-450 nm. (B) Significate differences were observed between deoxy HbSS and deoxy HbSS+196 μM Vox, HbSS+259 μM Vox, HbSS+384 μM Vox and HbSS+625 μM Vox. Those results suggest that increasing the concentration of PKR activator increases the oxygen affinity of HbSS as this is depicted by the reduction in the peak wavelength shift to that of HbAA

FIGS. 6(A-B) illustrate varying concentrations of aptamer induce peak wavelength shift. (A) Soret band peak wavelength shifts for oxygenated blood with aptamer deoxygenated blood with aptamer, untreated blood, and voxelotor treatment were compared in the wavelength range of 350-450. Increasing the concentration of aptamer from 0.2 dosages (B) Increasing the concentration of aptamer from 0.2 dosages to 0.25 induces a reduction in peak wavelength from 416 nm to 414 nm. Aptamer treatment reduced the peak wavelength to a level that was equivalent to that of HbAA which suggests that aptamer increases the hemoglobin oxygen affinity of HbSS and makes it equivalent to HbAA whole blood oxygen affinity.

FIGS. 7(A-D) illustrate rapid measurement of hemoglobin-oxygen dissociation by leveraging the Bohr effect and the Soret band bathochromic shift. (A) Sample analytes, purified hemoglobin (Hb), red blood cells (RBCs), or whole blood are prepared in a buffer with controlled pH. The analyte is then oxygenated at room air (162 mmHg) to form oxygenated Hb (Oxy-Hb). (B) Oxy-Hb analytes are mixed with sodium metabisulphite (Na₂S₂O₅) for 90 seconds to form partially deoxygenated Hb (Deoxy-Hb). (C) The optical absorption spectrum of a representative blood sample from an individual with sickle cell disease, readout acquired in the 350-750 nm range. The bathochromic shift from the 414 nm Soret band (light-blue highlighted) is measured. (D) The Soret band peak shifts from 414 nm to 430 nm peak upon full deoxygenation but halfway on partial deoxygenation. The bathochromic shift was systematically evaluated with partial deoxygenation and optical readout in the 390 nm to 450 nm range, which takes 30 seconds to acquire.

FIGS. 8(A-B) illustrate pO₂ reduction in whole blood depends on the Na₂S₂O₅ concentration and pH. pO₂ measured after 4 minutes in HbAA (A), and HbSS (B), (n=5) whole blood at pH of 6.9, 7.2, 7.4, and 8.0, at different independent Na₂S₂O₅ concentrations ranging from 0-0.08 M. At all pH values, pO₂ was at 160-162 mmHg without Na₂S₂O₅ and decreased to a range of 20-40 mmHg with increased Na₂S₂O₅ concentration.

FIGS. 9(A-I) illustrate peak shifts with deoxygenation for HbAA and HbSS differ with pH changes. Bathochromic shifts of purified HbAA and HbSS at pO₂ from 162-10 mmHg and pH of 6.9 (A), 7.2 (B), 7.4 (C), 8.0 (D), and 10.0 (E). (F) At 75 mmHg pO₂, HbAA vs. HbSS. (G) RBCs at pH 6.9. (H) Whole blood at pH 6.9. (I) At 75 mmHg pO₂, RBCs, and whole blood (***p<0.001), (**p>0.05) and (*p>0.05).

FIGS. 10(A-J) illustrate optical absorption variables correlate with p₅₀ (mmHg), sickle hemoglobin fraction (% HbSS), and Hb concentration (g dl⁻¹) at 75 mmHg pO₂ at pH 6.9. (A) Representative Hb-O₂ dissociation curves. (B) Bathochromic shift correlates with p₅₀. (C) HbSS bathochromic shift associates with sickle hemoglobin fraction in the sample. (D) Absorption intensity correlates with Hb concentration. (E) The area under the curve is negatively associated with peak wavelength, and (F) positively associated with FWHM. (G) HbSS vs. HbAA peak wavelength shift, (H) absorption intensity (I), the area under the curve, and peak FWHM (J).

FIGS. 11(A-C) illustrate optical absorption variables and peak wavelength shifts characterize Hb-O₂ dissociation differences. (A) Principal component analysis (PCA) of the absorption peak wavelength data were used to distinguish HbAA and HbSS, (B) PCA of the absorption peak wavelength data used to categorize sickle cell trait (HbAS=9), homozygous sickle cell disease (HbSS) on various treatments, hydroxyurea (Hu) HbSS=15, exchange transfusion (TF) HbSS=8, and treatment Naïve HbSS=20. (C) Peak wavelength shift for whole blood was used as a single variable to characterize HbAA, Hu HbSS, TF HbSS, Naïve HbSS, and HbAS.

FIGS. 12(A-H) illustrate circular Dichroism (CD) spectra of purified HbAA differ from that of HbSS at pH 6.9 and coincide with the peak wavelength shift differences. (A) Schematic structure of Oxy-Hb showing the position of heme. (B) Structural change of Deoxy Hb's heme porphyrin position. (C) CD spectra of Oxy HbAA and Oxy HbSS in 190-260 nm range. (D) CD spectra of Deoxy HbAA and Deoxy HbSS in 190-260 nm range. (E) CD spectra of Oxy HbAA and Oxy HbSS in 270-460 nm range. (F) CD spectra of Deoxy HbAA and Deoxy HbSS in 270-460 nm range. (G) CD spectra of Oxy HbAA and Oxy HbSS in 480-650 nm range. (H) CD spectra of Deoxy HbAA and Deoxy HbSS 480-650 nm range.

FIG. 13 illustrates increasing the hemoglobin concentration (mg/dl) did not affect the shifting phenomenon in the oxygen-hemoglobin dissociation assay. Increasing the concentration of Hb from 0.3-11.2 mg/dl did not affect the peak wavelength shift. For all Hb concentrations below the critical polymerization concentration of 34 g/dl, the peak wavelength of purified Hb was 420±0.2.

FIG. 14 illustrates robustness and repeatability of the oxygen-hemoglobin dissociation assay. Repeatability was determined from 20 tests comparing variances between 2 users. The peak wavelength shifts between the two users demonstrated strong repeatability (User1: HbSS: 421.6±0.24, HbAA: 414.9±0.11; User2: HbSS: 421.1±0.26, HbAA: 415.1±0.05) and coefficient of variance (COV)=0.05% for HbSS and 0.02% for HbAA).

FIG. 15 illustrates peak wavelength comparative analysis of the purified Hb, RBCs, and Whole blood upon deoxygenation. Comparative analysis is shown for 5 individuals with HbSS (sickle hemoglobin) and 3 individuals with HbAA individual (healthy hemoglobin) in deoxygenated state. The peak wavelength shifts for RBCs were found to be higher than purified Hb (p=0.001) and Whole blood (p=0.001) and purified Hb was higher than whole blood for the same patient (p=0.05). Peak wavelength shifts for HbAA are lower than that of HbSS.

### DETAILED DESCRIPTION

To facilitate the understanding of this invention, a number of terms are defined below. Terms defined herein have meanings as commonly understood by a person of ordinary skill in the areas relevant to the present invention. Terms such as “a”, “an”, and “the” are not intended to refer to only a singular entity but also plural entities and also includes the general class of which a specific example may be used for illustration. The terminology herein is used to describe specific aspects of the invention, but their usage does not delimit the invention, except as outlined in the claims.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation. “Optional” or “optionally” means that the subsequently described circumstance may or may not occur, so that the description includes instances where the circumstance occurs and instances where it does not.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

Embodiments described herein relate to a high throughput assay for screening hemoglobin oxygen affinity modifying agents. The assay takes advantage of differing hemoglobin (Hb) oxygenation and deoxygenation optical absorption spectra. It was found that under specific deoxygenation and pH levels, absorption spectra of sickle cell disease (SCD) Hb (e.g., HbS) samples had a larger bathochromic and hypochromic shift magnitude under deoxygenation at all pH levels compared to normal healthy blood containing HbA, where the higher the magnitude of the shift, the lower the oxygen affinity of HbS. We found that treated SCD samples with the oxygen modifying agents had reduced peak wavelength shift compared to untreated SCD samples, which means that the oxygen modifying molecules were effectively increasing the oxygen affinity. An increase in the concentrations of the oxygen modifying agents further induced a decrease in the peak wavelength shift. The optical absorption spectra of the SCD sample can be indicative of an oxygen affinity modifying agent's effect on hemoglobin oxygen affinity and particularly hemoglobin oxygen affinity of HbS of SCD samples and, thus, can be used to assess changes in the rate of deoxygenation with oxygen hemoglobin modifying drugs.

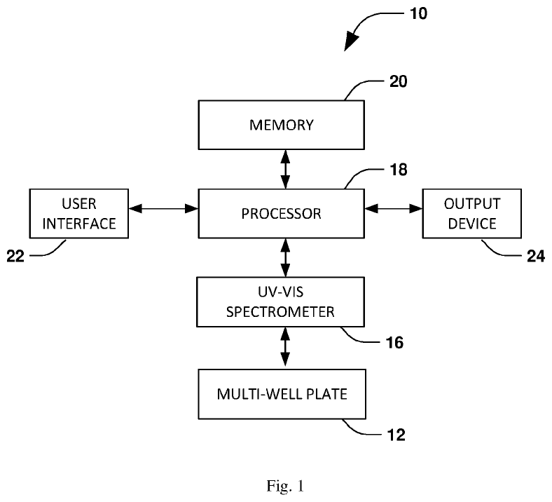

FIG. 1 illustrates a block diagram of an example of a high throughput assay 10 for determining at least one of hemoglobin oxygen affinity, rate of hemoglobin deoxygenation, and screening Hb oxygen affinity modifying agents. The assay 10 includes a multi-well plate 12, such as a 12-well, 24-well, 48-well, 96-well, 384-well plate, 1536 wells and the like, for high-throughput screening (HTS) Hb oxygen affinity modifying agents. By “high-throughput”, it is meant the screening of large numbers of candidate agents simultaneously for an activity or feature of interest.

The multi-well plate is configured to receive a plurality of hemoglobin, red blood cells, or blood samples that have been oxygenated and/or deoxygenated, at least one therapeutic agent, and a buffer solution. The hemoglobin, red blood cells, or blood samples, at least one therapeutic agent, and a buffer solution can be pipetted into the wells of the multi-well plate. Typically, an integrated robot system consisting of one or more robots transports assay-microplates from station to station for sample and reagent addition, mixing, incubation, and finally readout or detection. Generally, the multi-well plate can include standard multi-well plates used to study various biological endpoints under different interventions. The bottom of the well can be glass or an optically transparent material, such as an optically transparent plastic, to allow for UV-VIS spectroscopy of a plurality of hemoglobin, red blood cells, or blood samples placed within the well.

The assay further includes a UV-VIS spectrometer 16 that is configured to determine an optical signature of hemoglobin, red blood cells, or blood in each well that has been deoxygenated and a processor or computer processor 18 processor configured for comparing the determined optical signature to a control optical signature in each of the wells wherein differences between the determined optical signature and the control optical signature is indicative of the agents' effect on hemoglobin oxygen affinity.

In some embodiments of the assay, the processor is configured to determine differences of absorption spectra of oxygenated and deoxygenated hemoglobin, red blood, and/or blood obtained from the subject and compares the determined absorption spectra differences to a control value.

In some embodiments of the assay, the processor is configured to determine differences of absorption spectra by comparing a first optical absorption spectrum of oxygenated and/or deoxygenated hemoglobin, red blood cells, or blood with a second optical absorption spectrum of deoxygenated hemoglobin, red blood cells, or blood administered the oxygen affinity modifying agent.

In other embodiments of the assay, the processor is configured to determine differences in at least one of a bathochromic shift and/or hypochromic shift in peak wavelength from the first absorption spectra to the second absorption spectra.

In some embodiments of the assay, the magnitude of bathochromic shift in peak wavelength is indicative of at least one hemoglobin oxygen affinity or rate of hemoglobin deoxygenation.

In some embodiments of the assay, an increase in magnitude of biochromatic shift and/or hypochromic shift in peak wavelength compared to a control is indicative of decreased hemoglobin oxygen affinity or increased hemoglobin deoxidation.

In some embodiments of the assay, processor is configured to determine differences of area under a curve of and/or full width half maximum of peak wavelengths of the first absorption spectra and the second absorption spectra are indicative oxygen affinity modifying effect of the agent.

In some embodiments of the assay, the absorption spectra of the oxygenated and chemically deoxygenated hemoglobin are measured at the same pH from about 6.5 to about 7.0, preferably about 6.8 to less than 7.0, or more preferably, about 6.9.

In some embodiments of the assay, the hemoglobin, red blood cells, and/or blood is chemically deoxygenated by mixing the hemoglobin, red blood cells, and/or blood with an amount of chemical deoxygenant, such as sodium metabisulfite, effective to deplete oxygen from the hemoglobin.

The processor 18 typically receives and processes optical measurements that are performed by the UV-VIS spectrometer 16. Further typically, the processor 18 controls the acquisition of optical measurements that are performed by the UV-VIS spectrometer. The processor 18 communicates with a memory 20. A user (e.g., a laboratory technician) sends instructions to the computer processor via a user interface 22. For some applications, the user interface includes a keyboard, a mouse, a joystick, a touchscreen device (such as a smartphone or a tablet computer), a touchpad, a trackball, a voice-command interface, and/or other types of user interfaces that are known in the art. Typically, the computer processor generates an output via an output device 24. Further typically, the output device includes a display, such as a monitor, and the output includes an output that is displayed on the display. For some applications, the processor generates an output on a different type of visual, text, graphics, tactile, audio, and/or video output device, e.g., speakers, headphones, a smartphone, or a tablet computer. For some applications, user interface 22 acts as both an input interface and an output interface, i.e., it acts as an input/output interface. For some applications, the processor generates an output on a computer-readable medium (e.g., a non-transitory computer-readable medium), such as a disk, or a portable USB drive, and/or generates an output on a printer.

In some embodiments, the temperature of the multi-well plate may be controlled by a temperature control subsystem (not shown), which measures the multi-well plate temperature and if necessary, controls the ambient conditions to maintain a desired system temperature. Temperature subsystems may include any convenient temperature control protocol, including, but not limited to heat sinks, fans, exhaust pumps, vents, refrigeration, coolants, heat exchanges, Peltier or resistive heating elements, among other types of temperature control protocols.

In some embodiments, the UV-VIS spectrometer 16 can include broadband light source (such as a Tungsten Halogen bulb), monochromator that selects certain wavelengths from that broadband light source, a computer than can command the monochromator to select certain wavelengths, a collimating stage that accepts a sample and a detector that characterizes light intensity after it has passed a well of the multi-well plate.

In some embodiments, the memory 20 has instructions stored thereon, which when executed by the processor, cause the system to irradiate the sample with the desired wavelengths of light, determine a measured intensity of light at the desired wavelengths, and calculate the absorbance until a spectrum of absorbance vs wavelength covering the desired wavelength region.

In some embodiments, the computer readable medium includes instructions stored thereon for separating an absorption spectrum into a Rayleigh scattering contribution and an absorption contribution. In some cases, the instructions comprise: an algorithm for measuring an absorption spectrum (ii) an algorithm for generating a fit spectrum by fitting the absorption spectrum to a power function (iii) an algorithm for generating a difference spectrum by subtracting the fit spectrum from the absorption spectrum (iv) an algorithm for generating an adjusted spectrum by selecting points from the absorption spectrum for wavelengths wherein the difference spectrum is less than or equal to zero points from the fit spectrum for wavelengths wherein the difference spectrum is greater than zero (v) an algorithm for repeating steps (ii)-(iv) zero or more times, wherein the most recent adjusted spectrum is used in place of the absorption spectrum if the steps are repeated.

In some embodiments, the assay described herein can employ supervised machine learning. In some embodiments, supervised machine learning can detect difference in the generated or measured absorption spectra. For example, supervised machine learning can detect changes or differences in differences of a bathochromic shift and/or hypochromic shift in peak wavelength from generated or measured absorption spectra. In some embodiments, supervised machine learning can detect changes such as differences of area under a curve of and/or full width at half maximum of peak wavelengths of absorption spectra. In some cases, supervised machine learning can be used to classify samples Hb variant or concentration as well as hemoglobin oxygen affinity or rate of hemoglobin deoxygenation.

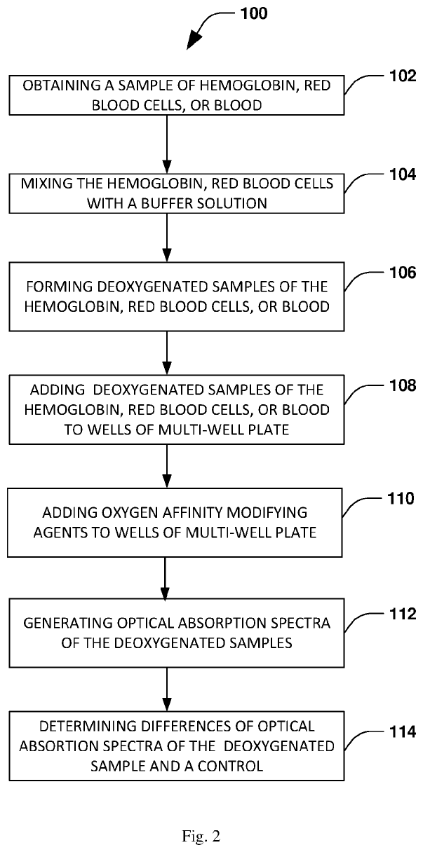

FIG. 2 illustrates a flow chart 100 showing a high throughput method for screening oxygen affinity modifying agents.

In the method at step 102, a sample of hemoglobin, red blood cells, or blood can be obtained. The sample can include whole blood, isolated red blood cells (RBCs), and purified hemoglobin lysed RBCs.

At step 104, the hemoglobin, red blood cells, and/or blood can be mixed with a buffer solution to provide a hemoglobin, red blood cell, or blood suspension with a pH that amplifies the Bohr effect of hemoglobin (Hb) including Hb variants, such as HbS. Any change in pH beyond the physiological range of 7.35 to 7.45 amplifies this Hb Bohr effect.

In some embodiments, the pH of the hemoglobin, red blood cell, or blood suspension can be adjusted with the buffer from a physiological blood pH of about 7.35 to 7.45 to a pH less or greater than physiological pH in a range of about 6.5 to about 9.0. For example, the hemoglobin, red blood cell, or blood suspension can be adjusted with the buffer from physiological blood pH to about 6.8 to less than 7.35 or greater than about 7.45 to less than 8.5, or preferably, about 6.9.

By way of example, whole blood, RBCs, and/or purified Hb can be mixed with pH 6.9 buffer solution and incubated for a duration of time at room temperature. The pH of the buffer suspensions can be checked after addition to the whole blood, RBCs, or Hb.

Following mixing of the Hb, RBCs, or blood with the pH buffer, at step 106, samples of the pH adjusted Hb, RBCs, or blood suspension can be oxygenated and/or deoxygenated to provide oxygenated and/or deoxygenated samples of the pH buffered Hb, RBCs, or blood suspension. For example, all samples can initially exposed to ambient air. The oxygen partial pressure (PO₂) which is 21% of atmospheric pressure (773 mmHg), can then normalized to 162 mmHg PO₂ during deoxygenation to provide deoxygenated samples of the Hb, RBCs, or blood suspension.

Samples of the Hb, RBCs, or blood suspension can also be deoxygenated chemically or enzymatically using, for example, sodium metabisulphite (Na₂S₂O₅), sodium dithionite (Na₂S₂O₄), or EC-oxyrase. For example, predetermined amounts of sodium metabisulphite can be mixed with the prepared samples of the Hb, RBCs, or blood suspension to provide deoxygenated samples with a gradual reduction in the oxygen partial pressure and deoxygenation levels.

At step 108, the oxygenated and/or deoxygenated mixed hemoglobin, red blood, and/or blood samples and buffer solution are added to wells of a multi-well plate by, for example, pipetting the mixed hemoglobin, red blood, and/or blood samples and buffer solution.

At step 110, at least one oxygen affinity modifying agent, such as voxelotor (previously known as GBT440), a Pyruvate Kinase Activator, and an aptamer anti-sickling agent, from a library of agents are added individual wells of the multi-well plate.

At step 112, optical absorption spectra of the oxygenated and/or deoxygenated Hb, RBC, and/or blood samples can be generated using a UV-VIS. The optical absorption spectra can include a generated optical absorption spectrum of oxygenated and/or deoxygenated Hb, RBCs, and/or blood suspension. The spectral rang of the absorption spectra can be from about 300 nm to about 800 nm with a resolution of, for example, about 1 nm or 2 nm. For example, during sample testing of either oxygenated and deoxygenated samples, samples can be analyzed in microplate wells using a spectroscopy microplate reader, such as Petromax Me2, (Molecular devices, San Jose, CA), over a spectral range of about 350 nm to about 750 nm, with a wavelength resolution of 2 nm and at a customized microplate well reading setting and at room temperature.

By way of example, deoxygenated samples were analyzed, to obtain reference signatures or control signature or control optical absorption spectra that included a Soret band (e.g., 380-480 nm) and two peaks in Q-band (e.g., 560-580 nm). All deoxygenated samples had their highest absorption oxygenated peak at 414 nm. Following that, the deoxygenated samples were analyzed across the same spectral range as the deoxygenated samples, but with Na₂S₂O₅ concentrations varying, for example, from 0.039 to 0.092M. The concentration of Na₂S₂O₅ used was corresponding to a decrease from 100-0 (mmHg) of pO₂ in samples.

In some embodiments, absorbance measurements are conducted at a substantially constant temperature. As such, the temperature during absorbance measurement changes by 5° C. or less, such as by 4.5° C. or less, such as by 4° C. or less, such as by 3.5° C. or less, such as by 3° C. or less, such as by 2.5° C. or less, such as by 2° C. or less, such as by 1.5° C. or less, such as 1° C. or less, such as by 0.5° C. or less, such as by 0.1° C. or less, such as by 0.05° C. or less, such as by 0.01° C. or less, such as by 0.005° C., such as by 0.001° C., such as by 0.0001° C., such as by 0.00001° C. or less and including by 0.000001° C. or less.

Following generation of the optical absorption spectra of the deoxygenated samples of Hb, RBCs, and/or blood, at step 114, the optical absorption spectra of the Hb, RBCs, and/or blood can be compared to a control absorption spectra of deoxygenated hemoglobin, red blood, and/or blood mixed with a similar buffer solution. The absorption spectra differences are indicative of the agent's effect on modulating hemoglobin oxygen affinity of the hemoglobin, red blood, and/or blood.

A “control value” or “appropriate standard” is a standard, parameter, value or level indicative of a known outcome, status or result (e.g., a known disease or condition status). A control value or appropriate can be determined (e.g., determined in parallel with a test measurement) or can be pre-existing (e.g., a historical value, etc.). For example, a control value or appropriate standard may be a bathochromic shift and/or hypochromic shift in peak wavelength, differences of area under a curve, and/or differences of the full width at half maximum of peak wavelengths obtained from a subject known to have a sickle cell disease, or a subject identified as being disease-free. In the former case, a lack of a difference between the measured differences in adsorption spectra and the differences in absorption spectra of an appropriate standard may be indicative of a subject having a disease or condition. Whereas in the latter case, the presence of a difference between the measured differences of absorption spectra and the differences of absorption spectra of the control value or appropriate standard may be indicative of a subject having a disease or condition.

The magnitude of a difference between a parameter, level or value the absorption spectra that is indicative of outcome, status or result may vary. For example, a significant difference that indicates a known outcome, status or result may be detected when the level of a parameter, level or value is at least 1%, at least 5%, at least 10%, at least 25%, at least 50%, at least 100%, at least 250%, at least 500%, or at least 1000% higher, or lower, than the appropriate standard. Similarly, a significant difference may be detected when a parameter, level or value is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than the level of the appropriate standard. Significant differences may be identified by using an appropriate statistical test. Tests for statistical significance are well known in the art and are exemplified in Applied Statistics for Engineers and Scientists by Petruccelli, Chen and Nandram Reprint Ed. Prentice Hall (1999).

In some embodiments of the method, the differences of the absorption spectra include at least one of a bathochromic shift and/or hypochromic shift in peak wavelength from the first absorption spectrum to the second absorption spectrum.

In some embodiments of the method, the magnitude of bathochromic shift in peak wavelength is indicative of at least one hemoglobin oxygen affinity or rate of hemoglobin deoxygenation.

In some embodiments of the method, an increase in magnitude of bathochromic shifty and/or hypochromic shift in peak wavelength is indicative of decreased hemoglobin oxygen affinity or increased hemoglobin deoxidation.

The function and advantage of these and other embodiments of the present invention will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### Example 1

This example relates to a 96-well high throughput assay optical absorption spectroscopic assay that can be used to assess the rate of oxygen release from hemoglobin.

### Method

Whole blood, purified hemoglobin, or washed red blood cells (RBCs) are mixed with a buffer solution at 6.8 or 8. Additional hemoglobin or RBCs to an acidic buffer changes the molecular conformational structure of hemoglobin. Determined volume of the analyte (including but not limited to whole blood, purified hemoglobin, or red blood cells (RBCs)) are mixed with buffer are added to each of the 12/96/384 well plates. The analytes are then chemically deoxygenated with a determined concentration of sodium metabisulphite an approach to deplete oxygen from hemoglobin molecules. The solution is analyzed using a spectroscopic microplate reader. The solution light absorption yields unique optical signatures of hemoglobin. The shift in the Soret band (414 nm absorption peak of hemoglobin) absorption spectral of hemoglobin can be used to assess the oxygen release and the extent of oxygen dissociation from hemoglobin. The results from the 96 well plates are obtained between 6-8 minutes.

### Results

We have used a validated bench top absorption spectroscopy 96 well plate analyzer to perform proof of concept on nearly 3 oxygen modifying molecules voxelotor (previously known as GBT440), a Pyruvate Kinase Activator, and an aptamer anti-sickling agent using 5 patient blood samples with sickle cell disease (SCD) and healthy patient samples (HbAA). Our preliminary results from 10 clinical samples show: At oxygenation (HbAA) samples exhibit a Soret peak wavelength at 414 nm, this was the same for SCD samples. However, under deoxygenation, the Soret peak wavelength shifts to 416 nm for HbAA and 426 nm for SCD, those results infer that HbAA samples have a higher oxygen affinity and SCD samples have a low oxygen affinity thus higher tendance of oxygen release from hemoglobin seen with a higher Soret ban peak wavelength shift.

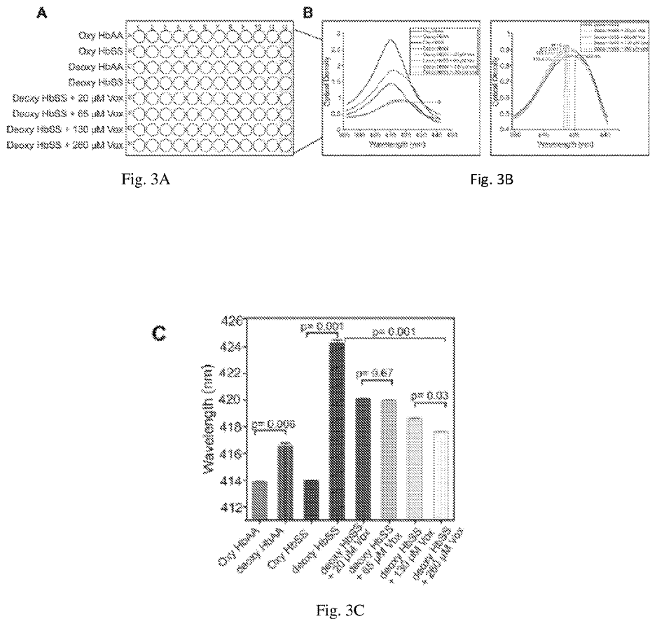

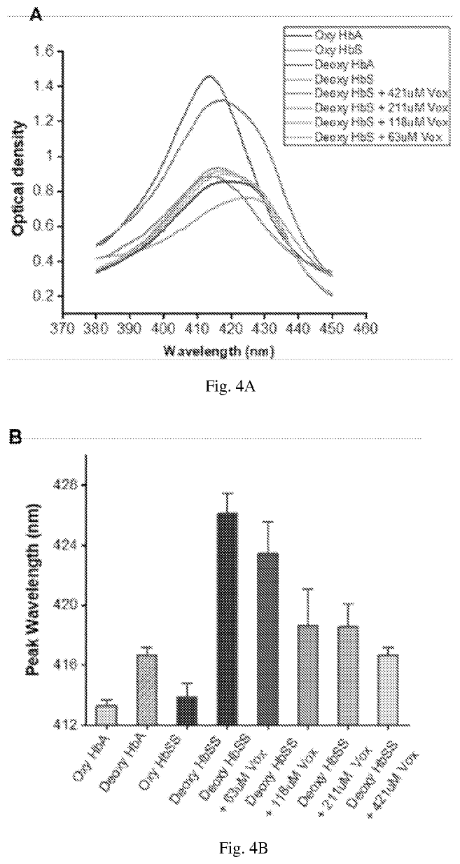

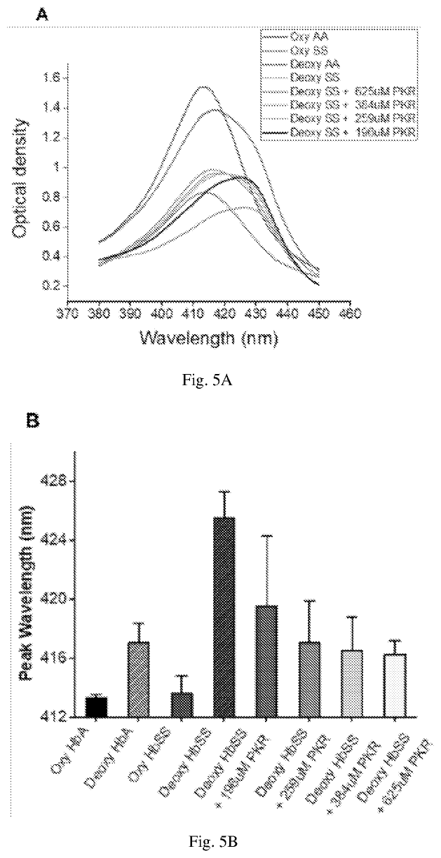

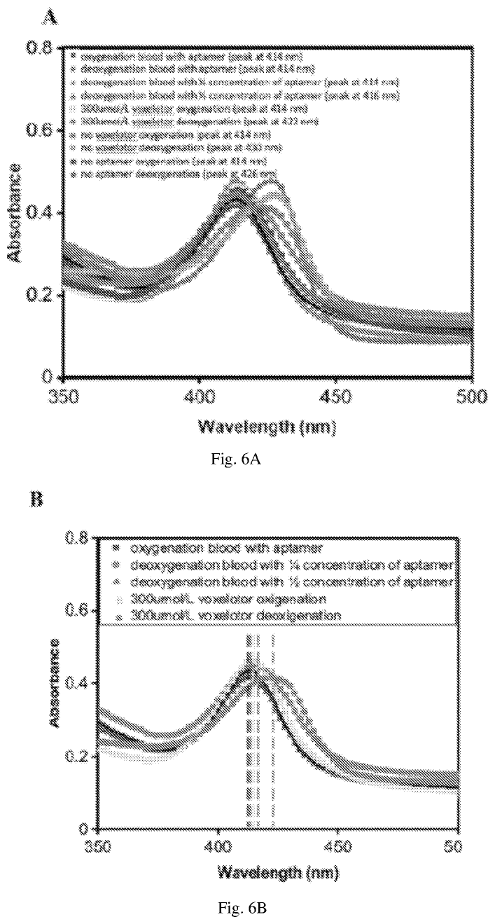

SCD samples were incubated with voxelotor (FIGS. 3 & 4), Pyruvate Kinase Activator (FIG. 5), and aptamer (FIG. 6) at varying concentrations for 3 hours, 5 hours, and 2 hours respectively. When tested for the peak wavelength shift, in a 96-well plate, we found that treated SCD samples with the oxygen modifying molecules had reduced peak wavelength shift compared to untreated SCD samples, which means that the oxygen modifying molecules were effectively increasing the oxygen affinity. An increase in the concentrations of the oxygen modifying molecules further induced a decrease in the peak wavelength shift. (FIG. 3-6).

### Example 2

Herein, we take advantage of the unique optical shift of Hb's Soret band and combine it with partial chemical deoxygenation to optically measure Hb-O₂ dissociation under physiologically acidic conditions at pH 6.9. First, we harnessed the acidic pH environment to facilitate rapid chemically induced partial deoxygenation of Hb (increased Bohr effect), to reduce Hb deoxygenation time down to 90 seconds. We have shown that, during this process, the conformational changes in Hb's tertiary structure result in significant shifts in the optical absorption spectra which concede with Circular Dichroism (CD) changes. This new rapid Hb-O₂ dissociation assay can be completed within 2 minutes with the following two steps: (1) mixing 1 part of the sample (whole blood, RBCs, or pure Hb) with 300 parts of 6.9 pH buffer and 0.052 M sodium metabisulfite (Na₂S₂O₅) which takes approximately 90 seconds per sample, and (2) adding the sample into a cuvette or a 96-well microplate for a light absorbance readout using a standard spectrophotometer (i.e., plate reader) in the visible range of 390 nm to 450 nm that includes the characteristic Hb Soret band, which takes approximately 30 seconds per sample.

In addition to significantly expediting the Hb-O₂ dissociation measurement, this new assay allowed for a systematic comparison of healthy Hb (HbAA) and sickle Hb (HbSS), revealing previously unknown variations in the absorption spectra of these two variants in partially deoxygenated states. We examined spectral differences in purified forms of HbSS and HbAA, as well as in intact RBCs and whole blood samples. Our findings show that the absorption spectra of HbAA and HbSS were identical in the oxygenated state and at near-neutral pH. However, under specific, time-controlled deoxygenation levels and pH conditions, the Soret band (at 414 nm wavelength) of HbAA and HbSS exhibited distinct behaviors. This distinction allowed us to differentiate between the two Hb variants based on their O₂ dissociation. HbSS displayed a significantly more rapid and pronounced shift of the Soret peak wavelength to the right (bathochromic shift) compared to HbAA. We assessed the utility of this assay as an optical indicator of treatment status by comparing it with existing clinical laboratory tests for Sickle Cell Disease (SCD) and by evaluating the impact of treatments (hydroxyurea, exchange transfusion) that modify hemoglobin composition in blood as well as Hb-O₂ oxygen affinity and dissociation.

Leveraging this newly discovered approach, we evaluated bathochromic shift as an optical marker for O₂ dissociation and compared it with p₅₀ values obtained using a Hemox analyzer on the same samples. As with p₅₀ values, the extent of the bathochromic shift in HbSS notably correlated with the percentage of sickle hemoglobin in the blood samples. We demonstrated the practical application of this technique in screening for sickle hemoglobin (HbS) in whole blood samples from individuals with SCD and healthy individuals. Additionally, this method allowed us to differentiate between various SCD genotypes and patients undergoing different therapies. This technique can transform the development and clinical testing of Hb-O₂ affinity-modifying drugs and gene-based therapies.

### Materials and Methods

### Materials

Phosphate buffer solutions of pH 6.9, 7.2, 7.4, 8.0, and 10.0 were purchased from Fisher Scientific (Pittsburgh, PA). All buffer solutions were stored at room temperature. Sodium metabisulfite (Na₂S₂O₅) was purchased from Sigma Aldrich (St. Louis, MO). Nunc polystyrene 90 microwell plates with nontreated surface and a flat bottom and lid were purchased from Thermo Fisher Scientific (Waltham, MA).

### Blood Samples and Human Subjects

Blood samples were collected from healthy donors and individuals with SCD as part of standard clinical care. We received ethical approval from the University Hospitals of Cleveland Medical Center's Institutional Review Board (IRB) and Joint Clinical Research Ethics Committee in Kampala, Uganda, for the Naïve SCD group. All participants in the study provided written informed consent. When collected, blood samples were de-identified, kept at 4 degrees Celsius, and processed within 6 hours of receipt. Samples were collected in EDTA-containing vacutainer tubes and separated into two groups: HbAA from healthy donors, HbSS samples from individuals with SCD, and HbAS blood samples with individuals that have heterozygous sickle cell trait (HbAS). Hemoglobin profiles of samples were verified using the reference standard high performance liquid chromatography (HPLC, VARIANT™ II, Bio-Rad Laboratories, Inc., Hercules, California). The results were presented as a percentage of HbS, A2, and F; the remainder was considered HbA since the values were normalized to 100%. Intracellular hemoglobin concentration for all blood samples was measured under normoxia with complete blood count (CBC, Hemavet 950FS, Hematology System, Draw Scientific Inc.; Miami, Florida).

### Sample Preparation

Three independent sample preparations were performed to test different hypotheses. (1) Whole blood, (2) isolated intact red blood cells (RBC), and (3) purified hemoglobin (lysed samples).

For whole blood preparation, 75 μl of whole blood was mixed with pH 6.9 buffers at a ratio of 1:300 and incubated for 10 minutes at room temperature before deoxygenation.

For intact RBC sample preparation, whole blood was centrifuged at 500 g for 10 minutes in a microcentrifuge (model 21r; Thermo Scientific, Waltham, MA). The plasma and buffy coat were removed via aspiration. The RBCs were washed thrice with phosphate-buffered saline (PBS) at pH 7.4 (Gibco, Thermo Scientific, Waltham, MA). Next, 45 μL of the washed RBC suspension was diluted with a pH 6.9 buffer solution until the baseline hematocrit of 0.2% was achieved. Before deoxygenation, RBCs were incubated for 10 minutes in a pH 6.9 buffer solution at room temperature.

For purified hemoglobin preparation; lysed samples were prepared. Hemolysis was carried out by sonication for 30 seconds (Hemex Health; Portland, OR). Following hemolysis, the lysed cell suspension was added to various pH buffer solutions and centrifuged at 2000 g for 1 hour. Three hundred μl of the cell lysate was removed, and the supernatant was dis-carded. Cell lysates were diluted in 700 μl of the specific pH buffer solution and centrifuged at 2000 g for 1 hour. The 1 ml of pH buffer solution and cell lysate were filtered through a 0.02 μm Millipore membrane. The hemoglobin concentration in the lysed sample was determined using the hemoglobin cyanide (HiCN) method and measured with a cuvette spectrophotometer at 540 nm. Each purified lysed sample was diluted with different pH solutions (pH 6.9, 7.2, 7.4, 8.0, and 10.0) to achieve a hemoglobin concentration of 2.8 mg dl⁻¹, determined as the baseline for lysed sample analysis. Methemoglobin was evaluated in blood samples, and if the optical spectra included a 635 nm band (methemoglobin peak), the sample was not included in our data analysis.

### pH Measurements for the Buffer and Samples

The pH of the buffer solutions was checked before and after in addition to the prepared samples with an acumen AE150 pH meter (Fisher Scientific, Waltham, MA). Using the same pH meter, we also measured the pH of a mixture of buffer and whole blood samples before and after adding Na₂S₂O₅.

### Oxygenation and Deoxygenation of the Samples

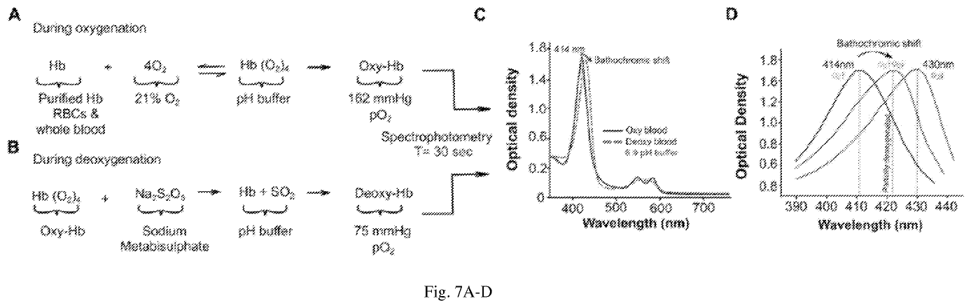

All samples were initially exposed to ambient air for 600 seconds. The oxygen partial pressure (pO₂), 21% of atmospheric pressure (773 mmHg), was then normalized to 162 mmHg PO₂ during oxygenation (FIG. 7A). Sodium metabisulphite (Na₂S₂O₅) was used to deoxygenate the samples chemically (FIG. 7B). Predetermined amounts of sodium metabisulphite were mixed with 2 ml of the prepared samples for 90 seconds using a vortexer (Scientific Industries, Bohemia, NY). The concentrations of Na₂S₂O₅ used with purified hemoglobin were 0.039 M, 0.046 M, 0.053 M, 0.059 M, 0.066 M, 0.079 M, and 0.082 M, which corresponded to a gradual reduction in the oxygen partial pressure in the samples. For whole blood and RBC analyses, we used 0.053 M of Na₂S₂O (17-fold more molecules than Hb molecules in solution) to achieve partial deoxygenation (Table 1). A blood gas analyzer (Nova Starter Pro, Nova BioMed, Boston, Massachusetts) was used to confirm the deoxygenation levels at those Na₂S₂O₅ concentrations.

TABLE 1
Comparison of Na2S2O5 molecules, Hb molecules, and O2 in buffer

| | | |
|---|---|---|
| Hemoglobin (Hb) and oxygen (O₂) molecules | | |
| Total Hb molecules used in phosphate buffer = | 0.1 | g/ml |
| Each Hb molecule weights = | 64500 | g/mol |
| Thus, Hb moles per ml of phosphate buffer (0.1/64500) = | 1.55 × 10⁻⁶ | mol/ml |
| Number of hemoglobin molecules per mole = | 6.02 × 10²³ | mol⁻¹ |
| Thus, total Hb molecules per ml of buffer (6.02 × 10²³*1.55 × 10⁻⁶) = | 9.34 × 10¹⁷ | molecule/ml |
| Each Hb molecule contains 4 O₂ molecules. | | |
| Thus, total O₂ molecules per ml in buffer (9.34 × 10¹⁷ *4) = | 3.78 × 10¹⁸ | molecule/ml |
| Na₂S₂O₅ molecules | | |
| Total Na₂S₂O₅ molecules used in phosphate buffer (0.053M) = | 0.02 | g/ml |
| Each Na₂S₂O₅molecule weights = | 190.107 | g/mol |
| Thus, Na₂S₂O₅ moles per ml of buffer (0.02/64500) = | 1.05 × 10⁻⁴ | mol/ml |
| Number of Na₂S₂O₅ molecules per mole = | 6.02 × 10²³ | mol⁻¹ |
| Thus, total Na₂S₂O₅ molecules per ml of buffer (6.02 × 10²³*1.05 × 10⁻⁴) = | 6.34 × 10¹⁹ | molecule/ml |

Ratio of Na₂S₂O₅ molecules:oxygen molecules per ml = 17

### Hemoglobin-Oxygen Dissociation Assay

During testing with either oxygenation or deoxygenation, samples were analyzed in three microplate wells using a spectroscopy microplate reader Spectramax M2e (Molecular Devices, San Jose, CA), over a spectral range of 350-750 nm, with a wavelength resolution of 2 nm and at a customized microplate well reading setting at room temperature. Each acquisition lasted 30 seconds, for each microplate well for a spectral range of 380-450 nm and 90 seconds for 350-750 nm. The sequence of the assay is as follows: (1) initially, the fully oxygenated samples were analyzed to obtain reference signatures that included a Soret band (390-450 nm) and two peaks in Q-band (560-580 nm). All oxygenated samples had their highest oxygenated absorption peak at 414 nm (FIG. 7C). (2) The deoxygenated samples were analyzed across the same spectral range as the oxygenated samples but with Na₂S₂O₅ concentration at 0.053 M. The concentration of Na₂S₂O₅ used corresponded to a decrease from 75 mmHg of pO₂ in samples. The spectra of deoxygenated samples yielded two distinct peaks with pO₂ reduction (FIG. 7C). The most prominent Hb peak at 414 nm in the oxygenated spectral range shifted, and the bathochromic shift varied depending on the type of sample under analysis and the pH buffer added, while the two peaks in the Q band range converged into a single peak at 560 nm (FIG. 7C). At pO₂ reduction of 75 mmHg in the sample, the spectral analysis yielded three different peaks; the bathochromic shift from 414 nm and the two peaks at 540 nm and 570 nm with a reduced gap between while the hump still present. (3) For each sample and at concentration of 0.053 M Na₂S₂O₅, the bathochromic wavelength shift and intensity were assessed for further analysis (FIG. 7D).

### Oxygenated and Deoxygenated Absorption Spectra

Oxygenated and deoxygenated spectra were obtained and processed in SoftMax Pro 6.3. Individual peaks, intensities, and peak wavelength shifts were identified from the spectra through an automated peak and intensity search (FIG. 7D). Using SpectraGryph 1.2, the Soret peak was baselined, smoothed, and examined. The area under the peak (380-460 nm) and full width at half maximum (FWHM) were then calculated from the normalized Soret peak (FIG. 7D).

### Circular Dichroism Spectra of Purified Hemoglobin at pH 6.9

The circular dichroism (CD) spectra, like the absorption spectra in the Soret and visible range, reveal the molecular structure of the hemoglobin globin and heme groups. To confirm the effect observed in our assay, we performed circular dichroism on oxygenated purified hemoglobin (OxyHbS, OxyHbA) and deoxygenated purified hemoglobin (DeoxyHbS, DeoxyHbA) at 75 mmHg and at pH 6.9 for HbAA and HbSS samples. CD spectra were collected between 190 and 650 nm wavelength on a Jasco J-1500 Spectropolarimeter with a 1 mm quartz cuvette and a scan rate of 4 seconds. The recorded spectra were background subtracted, accounting for a pH 6.9 buffer. The data was baselined and smoothed in the Spectra Manager software (Release 2018; Version 2.15.01).

### Statistical Analysis

We used Minitab software (Release 2021, Version 20; Minitab) for the statistical analysis. Mann Whitney U-test was performed to compare spectral variables between the HbAA and HbSS groups. Pearson's correlation analysis assessed the correlation between spectral variables; P<0.05 was considered statistically significant. Unless otherwise noted, results were expressed as means±standard error of the mean (SEM). Four optical variables (peak wavelength shift, intensity, FWHM, and area under the peak) data from HbAA, HbSS, and HbAS blood samples were pooled and subjected to two-dimensional principal components analysis (PCA) using R Studio (Release 2021; 4.1.1) independently. The first and second principal components were PC1 and PC2, respectively. The two components were clustered and visualized to represent the HbSS, HbAS and HbAA samples.

### Results

Sodium Metabisulphite (Na₂S₂O₅) Induces Hb-O₂ Dissociation and Oxygen Partial Pressure (pO₂) Decrease in a Dose- and pH-Dependent Manner

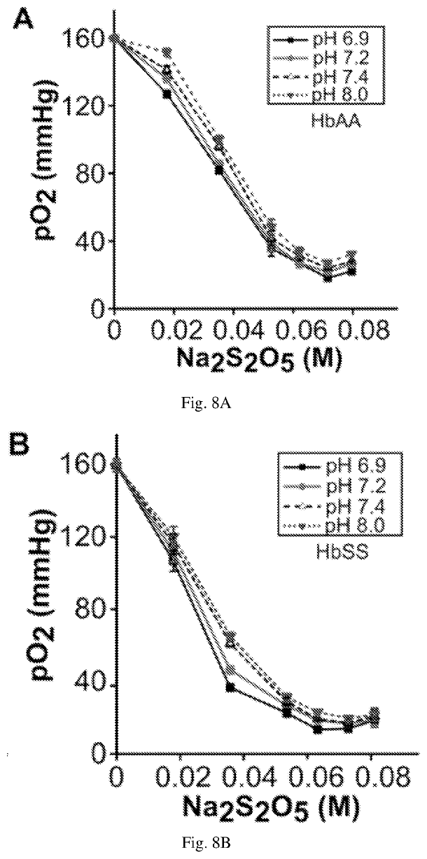

We used sodium metabisulphite (Na₂S₂O₅) to deoxygenate purified hemoglobin, intact RBCs, or whole blood (FIGS. 7A & B). To quantify the effect of Na₂S₂O₅ on deoxygenation, we measured the oxygen partial pressure (pO₂) in both HbAA and HbSS whole blood samples reconstituted with a range of Na₂S₂O₅ concentrations from 0 to 0.08 M at pH values of 6.9, 7.2, 7.4, 8.0, and 10.0. At all pH levels, the pO₂ levels of both HbAA and HbSS whole blood samples were determined to be 162 mmHg before the addition of Na₂S₂O₅ (FIG. 8). The pO₂ levels of both HbAA and HbSS whole blood samples decreased with increasing Na₂S₂O₅ concentration from 0.039 M to 0.059 M (FIGS. 8A & B). Under all Na₂S₂O₅ concentrations, the pO₂ of both HbAA and HbSS whole blood decreased with a decrease in pH (FIGS. 8A & B). HbSS whole blood demonstrated lower pO₂ levels compared to HbAA whole blood at the same Na₂S₂O₅ concentration and pH value, except at zero Na₂S₂O₅ concentration (max. pO₂=162 mmHg) and 0.053 M (min. pO₂ around 10 mmHg) (FIG. 8B). The 0.053 M concentration of Na₂S₂O₅ leads to partial deoxygenation of hemoglobin, where the number of Na₂S₂O₅ molecules are more than 17-fold of available oxygen molecules in solution (Table 1). Na₂S₂O₅ scavenges soluble oxygen in an aqueous buffer but does not fully deoxygenate oxyhemoglobin. At elevated concentrations of Na₂S₂O₅, corresponding to high levels of deoxygenation or pO₂ close to 30 mmHg, the curve plateaus (FIG. 8). This occurs because, at these concentrations, both HbAA and HbSS exhibit similar maximum oxygen dissociation behaviors in these conditions. Together, these results demonstrate that: (1) increase in the concentration of Na₂S₂O₅ and decrease in pH together lower pO₂ levels in blood samples, and (2) pO₂ levels in HbSS whole blood samples are subjected to a greater magnitude of decrease than HbAA whole blood samples when treated with the same concentration of Na₂S₂O₅.

The Bathochromic Shift of Hemoglobin with Oxygen Dissociation Depends on the pH

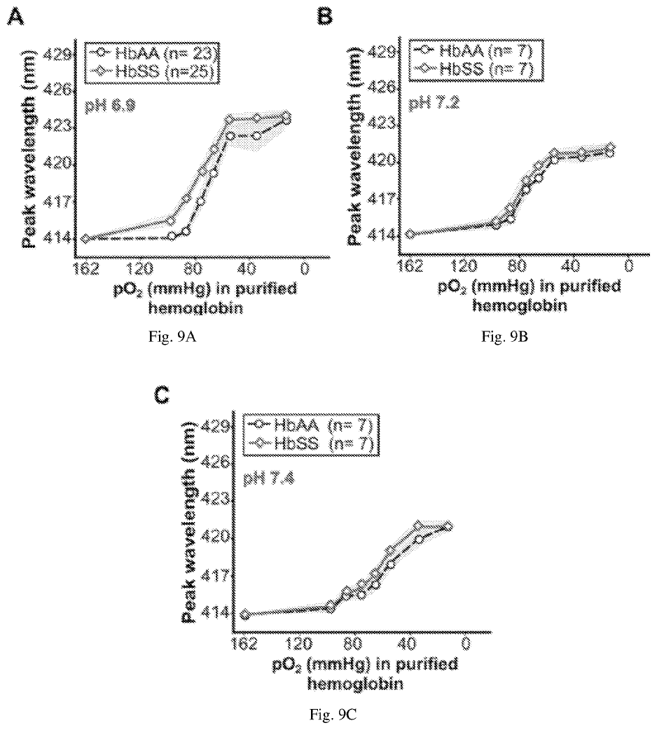

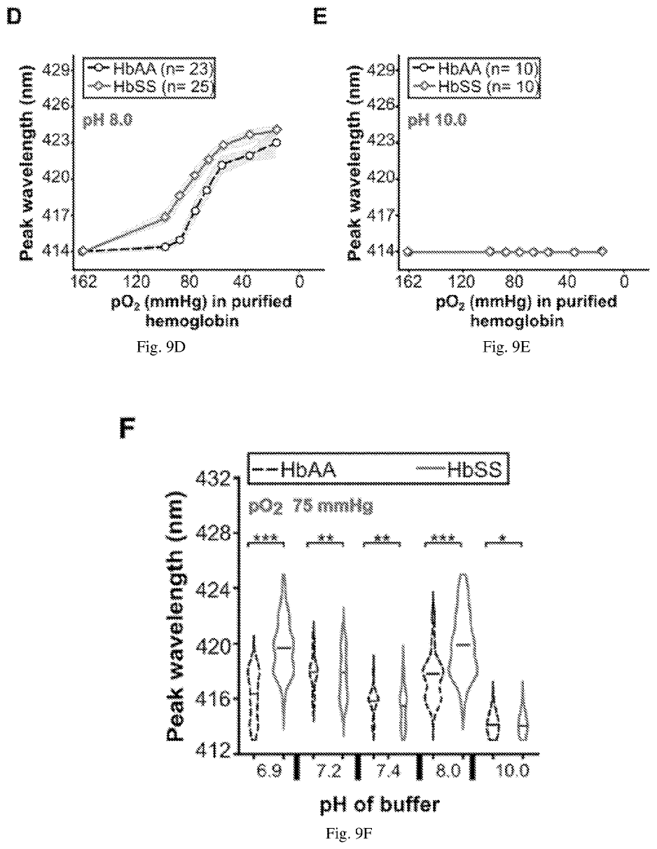

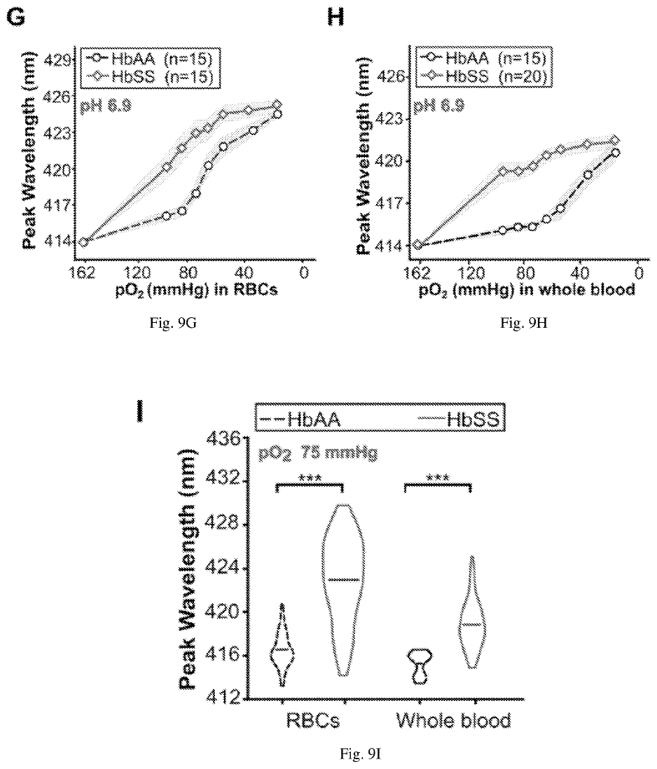

At all pH values, fully oxygenated (162 mmHg) HbAA and HbSS had the same peak wavelengths of 414 nm (FIG. 9). Decreasing the pO₂ from 162 to 20 mmHg induced peak wave-length shifts in both HbAA and HbSS purified hemoglobin at all pH values (FIG. 9A-D) except pH of 10.0 (FIG. 9E). At partial deoxygenation (pO₂=75 mmHg), significant differences in peak wavelength shifts between HbAA and HbSS were found at pH=6.9 (FIG. 9F) (p<0.001) and pH=8 (p<0.001). At pH=7.2 (FIG. 9F) (p>0.05) and pH>7.4 (FIG. 9F) (p=0.72), there were no significant changes in peak wavelength shifts between HbAA and HbSS. At pH 6.9, RBCs and whole blood followed the same trend as purified Hb (FIGS. 9G & H). These results demonstrate that pH influenced the bathochromic shift's magnitude for HbAA and HbSS purified hemoglobin. For HbAA and HbSS purified hemoglobin, the highest magnitude of bathochromic shift was obtained at pH 6.9, followed by pH 8.0. At all pH values except pH 10, HbSS had a greater magnitude of bathochromic shift compared to that of HbAA.

For the RBCs and whole blood samples, we focused our analyses on pH 6.9 because the highest peak wavelength difference between HbAA and HbSS purified hemoglobin was obtained at this pH value. The variations of bathochromic shift shown for purified hemoglobin were also evident for RBCs (FIG. 9G) and whole blood (FIG. 9H). At 75 mmHg pO₂, bathochromic shifts were significantly different between HbAA and HbSS whole blood samples (FIG. 9I) (***p<0.001) and HbAA and HbSS whole blood samples (FIG. 9I) (***p<0.001) were significantly different. Compared to the bathochromic shift of purified hemoglobin for HbAA and HbSS at pH 6.9, RBCs had the largest magnitude of the bathochromic shift, followed by purified hemoglobin and whole blood samples. Bathochromic shift summaries are shown in Table 2. HbSS RBCs and whole blood had a larger bathochromic shift than normal RBCs and whole blood. The findings demonstrate that the bathochromic shift difference shown by purified hemoglobin HbAA and HbSS is also observable when using RBCs and whole blood analytes.

TABLE 2
Peak wavelengths of hemoglobin analytes at pH 6.9 and pO2 = 75 mmHg

| Peak wavelength | | | |
|---|---|---|---|
| | HbAA (Sample size) | HbSS (Sample size) | P-value |
| Purified hemoglobin | 417.2 ± 0.78 (n = 23) | 420.3 ± 0.52 (n = 25) | P = 0.009 |
| RBCs | 418.0 ± 0.56 (n = 15) | 422.9 ± 0.63 (n = 15) | P = 0.002 |
| Whole blood | 415.3 ± 0.23 (n = 15) | 419.6 ± 0.48 (n = 20) | P = 0.001 |

HbAA: healthy hemoglobin.
HbSS: homozygous sickle cell disease hemoglobin.
Mann Whitney Non-parametric test was used to calculate p-values.
Data is reported as mean ± SEM.

Whole Blood Optical Absorption Parameters Demonstrate a Correlation with Hemoglobin Oxygen Affinity (p₅₀), Sickle Hemoglobin Fraction, and Hemoglobin Concentration

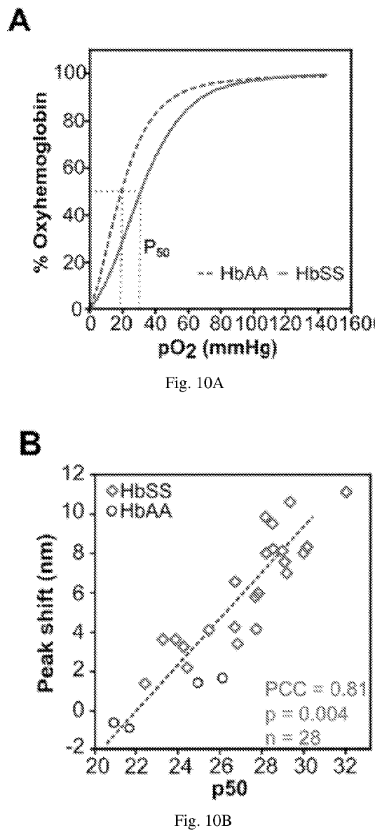

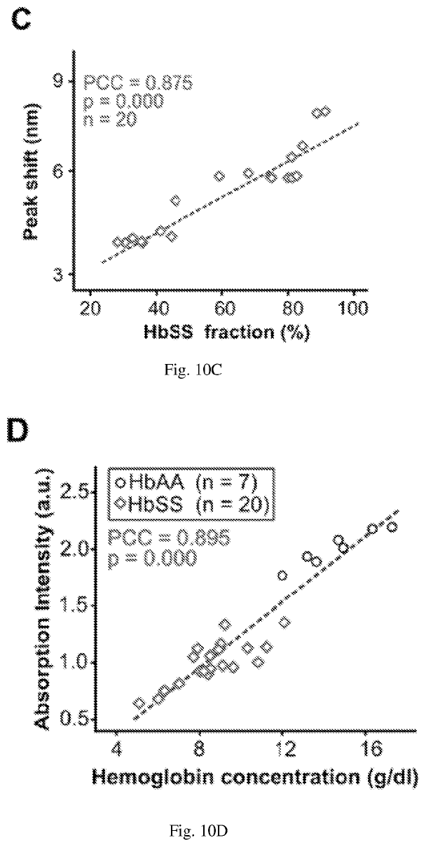

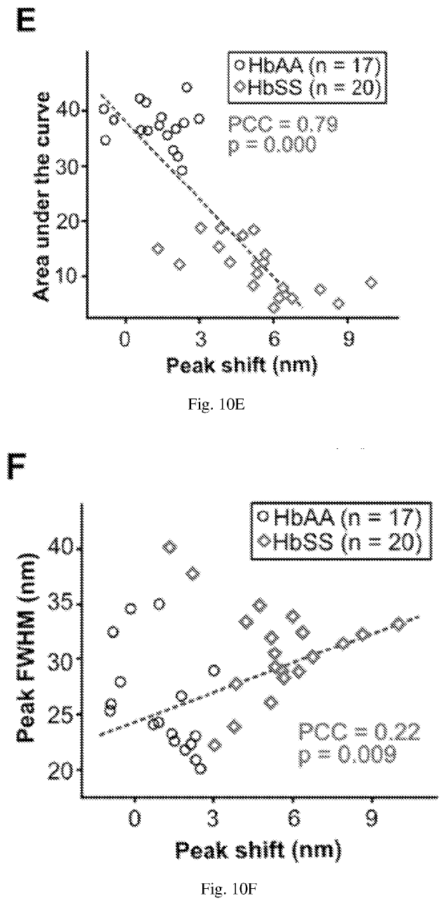

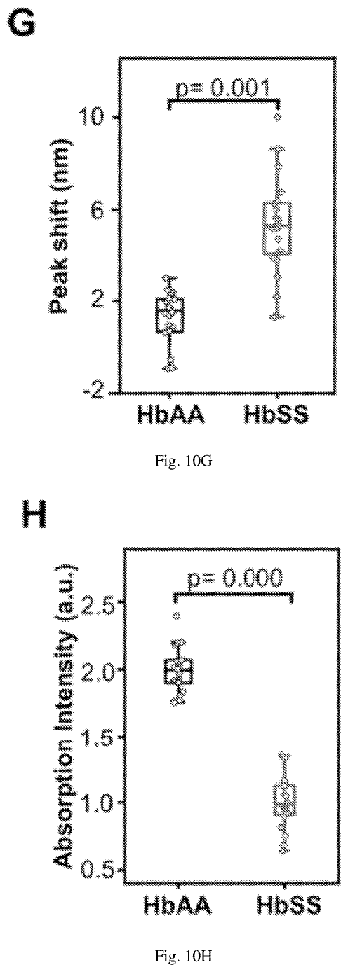

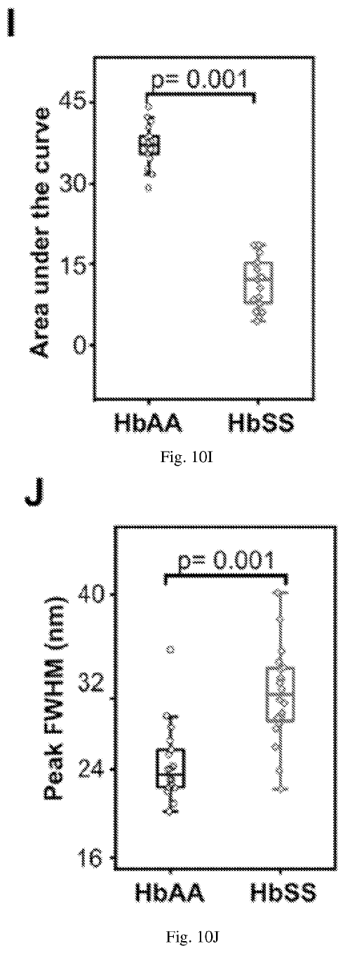

Aside from obtaining the measurable optical variables and examining their confounding effects, it was also critical to study the correlation between bathochromic shift and absorption intensity with p₅₀, HbS fraction, and Hb concentration (FIG. 10). HbAA and HbSS whole blood bathochromic shifts were associated with the changes in hemoglobin oxygen affinity as measured by p₅₀ value (FIGS. 10A & B) from the ODCs obtained using Hemox analyzer for each whole blood sample at pH 7.681.

HbSS whole blood bathochromic shifts at pH 6.9 and pO₂ of 75 mmHg were highly associated with the changes in the percentage of hemoglobin S (HbS %) obtained using High-Performance Liquid Chromatography (HPLC) for each whole blood sample at pH 6.9 and pO₂ 75 mmHg (FIG. 10C) (PCC=0.875, p=0.000, n=20). The absorption intensity for whole blood samples at pH 6.9 and 28 mmHg pO₂ (FIG. 10D) was strongly and positively associated with the concentration of hemoglobin (g dl⁻¹) obtained by the standard Complete Blood Count (CBC) for both HbAA and HbSS whole blood samples (PCC=0.89, p=0.000, HbSS (n=20), HbAA (n=7)). At 75 mmHg pO₂ (FIG. 10E), the area under the shifted Soret peak was significantly and inversely associated with a bathochromic shift in both HbAA and HbSS whole blood (PCC=−0.79, p=0.000, HbSS (n=20), HbAA (n=17)) (FIG. 10G). Full width at half max (FWHM) were significantly but weakly linked with peak wavelength shift for both HbAA and HbSS whole blood samples (PCC=0.22, p=0.009, HbSS (n=20), HbAA (n=17)). Additional correlations between the variables and physiological importance are shown in Table 3.

TABLE 3
Correlations between optical variables and physiological relevance

| | | | | | Physiological | |
|---|---|---|---|---|---|---|
| | Peak | Peak | Area under | peak | Parameter | Physiological |
| | wavelength | intensity | the curve | FWHM | Correlated | Relevance |
| Peak | 1 | | | | Percentage of | Identification |
| wavelength | | | | | variant | of Variant SS |
| | | | | | hemoglobin | |
| | | | | | SS | |
| Peak | PCC = −0.46 | 1 | | | Blood | Determine the |
| intensity | p = 0.000 | | | | concentration | level of |
| | Negative | | | | of | Hemoglobin |
| | | | | | Hemoglobin | level and |
| | | | | | (g/dl) | Anemia |
| Area under | PCC = −0.76 | PCC = 0.94 | 1 | | Anemia (low | Identification |
| the curve | p = 0.000 | p = 0.000 | | | hemoglobin | of Anemia |
| | Negative | Positive | | | detection) | |
| Peak | PCC = −0.22 | PCC = −0.41 | PCC = −40.0 | 1 | Sample | Variation in |
| FWHM | p = 0.006 | p = 0.000 | p = 0.000 | | homogeneity | samples |
| | Negative | Negative | Negative | | | |

*PCC: Pearson Correlation Coefficient, p-values were obtained from linear regression model

Comparisons of the optical variables between HbAA and HbSS whole blood indicated that HbSS whole blood had a significantly higher magnitude of bathochromic shift and FWHM compared to HbAA whole blood (HbSS; 420.6±0.44, HbAA; 415.3.0±0.21), (p=0.001) (FIG. 10G), and (HbSS; 30.9±0.84, HbAA; 24.3±0.82) (p=0.001) (FIG. 10J) respectively. HbAA whole blood had a significantly higher peak intensity and area under the peak compared to HbSS whole blood (HbSS: 0.99±0.04, HbAA: 2.0±0.03), (p=0.000), (FIG. 10H) and (HbSS: 11.6±0.69, HbAA: 37.2±0.8) (p=0.001) (FIG. 10I) respectively. These results revealed that the magnitude of bathochromic shift could be used to predict the hemoglobin oxygen affinity (p₅₀) and percentage of HbS in SCD patient samples, absorption intensity for determination of the concentration of hemoglobin, and anemia status and FWHM to assess the homogeneity of the sample. When compared to HbAA samples, the homogeneity of HbSS whole blood was less consistent, as shown by greater standard deviation, since HbSS patients were on different therapies, leading to varying fractions of HbS.

### Optical Peak Wavelength Shifts and Other Optical Variable Differences are Driven by Differences in Hb-O₂ Dissociation of HbAA, HbAS, HbSS and Sickle Cell Patients Based on Treatment

Principal component analysis (PCA) included optical variables (bathochromic shift, peak intensity, FWHM, and AUC data set constructed from HbAA, HbSS, and heterozygous sickle cell traits (HbAS) whole blood samples (FIG. 11). PCA resolved the differences in our data set. Initially, two components accounted for 94.5% variability in the optical parameters data set, with the first component accounting for 77.96% of the total variation between HbAA and HbSS whole blood samples (n=20) (FIG. 11A). Secondly, two principal components accounted for 98.5% variability in the optical parameters data set, with the first component accounting for 86.56% of the total variation between heterozygous sickle traits (HbAS)=9 and HbSS patients on various treatments: hydroxyurea (Hu HbSS=15), exchange transfusion (TF HbSS=8) and those not on any treatment (treatment Naïve HbSS=20) (FIG. 11B). We evaluated the HbAA, HbAS, and HbSS patient whole blood samples stratified in the context of treatment by only peak wavelength variable under both oxygenated and deoxygenated conditions at 75 mmHg at pH 6.9. Bathochromic shift displayed significant differences for HbAA, HbAS, and HbSS patient whole blood samples and stratified based on treatment (hydroxyurea, transfusion) (FIG. 11C). No significant differences were seen for peak wavelength shift for oxygenated HbSS, HbAA, Hu HbSS, TF HbSS, and HbAS except for Naïve HbSS p=0.02 (FIG. 11C). The PCA clearly showed grouping when using the four variables which diverged from the peak wavelength. We conclude that optical absorption variables or the bathochromic shift can be used to screen Hb-O₂ affinity disorders and evaluate the efficacy of treatments that modify Hb-O₂ affinity and dissociation.

### CD Spectra of Purified Hemoglobin at pH 6.9 Reveals Differences in the Heme-Heme Interaction for Deoxygenated HbAA and HbSS

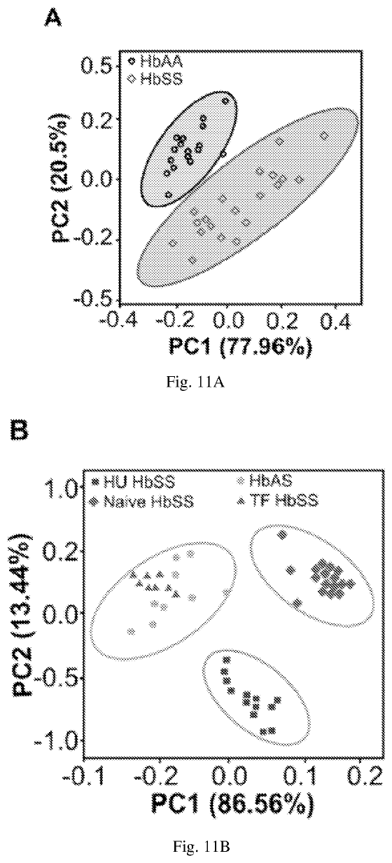

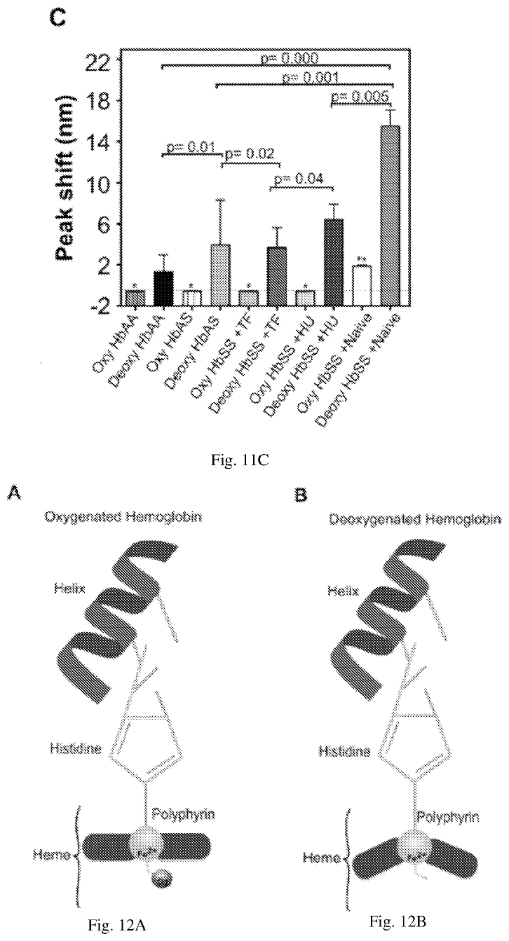

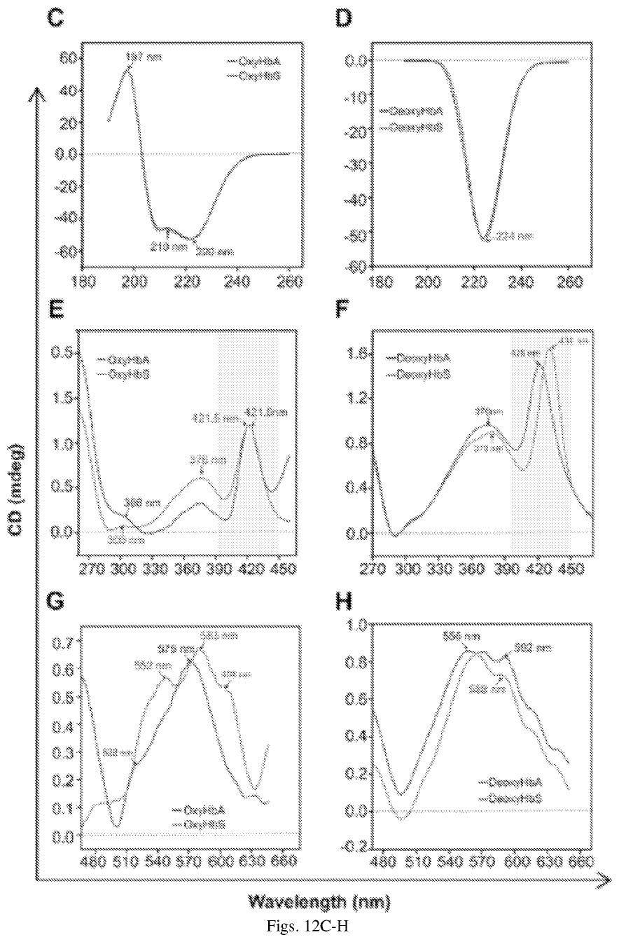

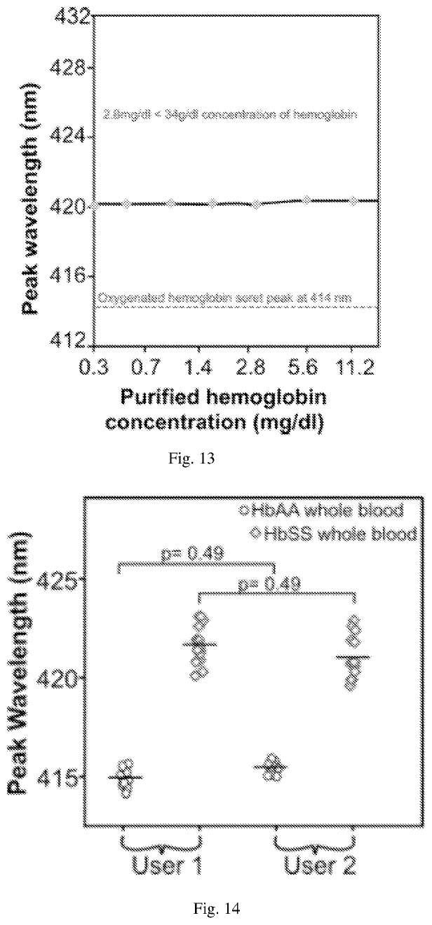

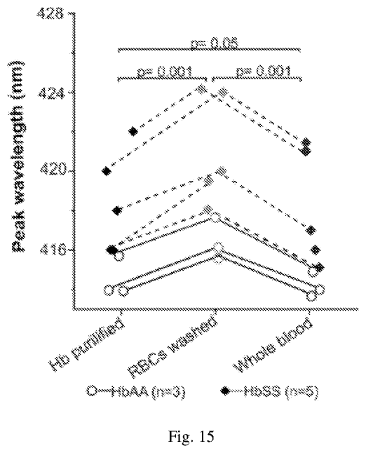

To understand the fundamental mechanism, behold the observed differences in the peak wavelength shift of HbAA and HbSS, the CD spectra of purified hemoglobin HbAA and HbSS were conducted under oxygenated and partially deoxygenated states (FIG. 12). Under Hb oxygenation, the heme iron atom rotates to become planar with the porphyrin rings, pulling the histidine and creating a broader-scale structural change in the protein (FIG. 12A). Under Hb deoxygenation, the iron atom is non-planar with the porphyrin rings due to its association with a histidine side chain (FIG. 12B). Heme-heme interaction arises from the transition between the Hb oxygenated (FIG. 12A), and Hb deoxygenated (FIG. 12B), which differ in the tertiary and quaternary structures. In the spectral region below 250 nm, which reveals quaternary conformations of Hb, Oxy HbAA and Oxy HbSS show identical spectra (FIG. 12C). Similar CD spectra were observed with deoxy HbAA and deoxy HbSS in that wavelength range (FIG. 12D). In the spectral range above 300 nm, the CD spectral results from the heme environment are caused by heme interactions between a and β chains of protein and with the aromatic residues of the globin. Slight differences were observed in the Oxy HbAA and Oxy HbSS (FIGS. 12E & G). Still, more significant differences were observed with Deoxy HbAA and Deoxy HbSS, particularly in the 260-470 nm wavelength range, which is close to the Soret band range (FIGS. 12F & H). The CD spectral differences between Deoxy HbAA and Deoxy HbSS in the Soret band corresponded to the differences we observed with peak wavelength shift. The Soret peak of deoxy HbSS was at 431 nm, while that of deoxy HbAA was 425 at partial deoxygenation. Those results provide a clear fundamental understanding of peak wavelength shift; the spectral differences we report for HbSS results from its unique heme environment as demonstrated with the shifted CD Soret band induced by heme interactions between α and β chains. The peak wavelength shift at partial deoxygenation and at pH 6.9 provide molecular conformational characterization of the tertiary Hb structure, reflecting variations in Hb-O₂ affinity.

### Hb Light Absorption in Oxygen Dissociation Assay

Net light extinction is a function of absorption and scattering, and light scattering is particularly sensitive to particle size and aggregation. While we cannot rule out scattering completely in the hemoglobin oxygen dissociation assay (Hb-O₂), its involvement is not likely to be as pronounced as absorption. The wavelength shift that we report holds regardless of Hb-O₂ dissociation measurements are conducted on Hb samples solubilized in solution (~5 nm particles), as RBCs (<10-micron particles), or whole blood (<10 microns and >10-micron particles). On the other hand, the magnitude of the shift was different between RBCs, whole blood, and purified Hb (Table 2), (FIG. 9), indicating that light scattering may affect the intensity of the wavelength shift phenomenon because Hb is at high concentrations in red cells, where sickling is possible upon deoxygenation. To ascertain that the effects we report mainly stem from absorption, we sought the wavelength shift in samples by gradually increasing the concentration of Hb. We have seen that the shifting phenomenon, along with the magnitude of the shift, did not change with increasing concentration (FIG. 7), implying that scattering is not a significant contributor to Hb-O₂ dissociation when the Hb is dissolved in the aqueous environment.

### Rigor and Reproducibility

The repeatability of the bathochromic shift was established by comparing variations between two users in repeated measurements of the same whole blood samples at ~20 mmHg pO₂ (FIG. 8). Two samples were used in this study (one HbAA and one HbSS). Each sample was evaluated five times, and the same sample was analyzed in three microwells throughout each test, resulting in 15 tests for each user. The peak wavelength shifts between the two users showed good repeatability ((Mean±SEM) (User1: HbSS 421.6±0.24, HbAA; 414.9±0.11; User2: HbSS 421.1±0.26, HbAA; 415.1±0.05) and coefficient of variance (COV)=0.05% for sickle and 0.02% for normal). Bathochromic shifts between User 1 and User 2 for both HbAA and HbSS whole blood (p=0.48) and (p=0.48) were not significantly different. These results indicate reproducibility and good precision of the assay.

We describe a new optical assay approach to quickly measure Hb-O₂ dissociation. This method enabled us to detect low oxygen affinity HbSS, stratify SCD patients based on treatment status, and measure Hb concentration for whole blood samples. We have demonstrated that, this new approach correlated with the reference standard p₅₀ value, which is a clinical indicator of Hb-O₂ affinity. In clinical practice, Hb's oxygen affinity is one of the most underutilized blood parameters, despite its critical significance in assessing blood oxygen availability and congenital disorders of erythrocytosis. However, the interest in p₅₀ measurement has been heightened with the accelerating development of Hb-O₂ affinity-modifying therapeutics for inherited Hb disorders. One of the major reasons why Hb oxygen affinity is frequently underutilized might be the unavailability of an alternative facile and accurate p₅₀ measurement method. We believe that the method presented here will significantly streamline the Hb-O₂ affinity assessment and improve its accessibility.

To our knowledge, this is the first time that the optical absorption spectra of HbSS's Soret band were shown to be significantly different from HbAA under specific deoxygenation and pH conditions, based on actual spectral bathochromic shifts. We have shown that the bathochromic shifts of HbAA and HbSS purified Hb were significantly different only at pH 6.9. No bathochromic shift was observable at pH 10.0. At higher pH, we noticed that HbSS has a similar O₂ dissociation to HbAA regardless of the hypoxia (pO₂) level at those conditions. We may counter that there is no bathochromic shift at alkali conditions, implying that O₂ is not rapidly released from the HbSS. Earlier attempts by researchers to alleviate SCD crises by alkalizing the blood can be deemed reasonable in this context.

SCD samples had a greater bathochromic shift magnitude under deoxygenation at all pH levels than normal healthy blood. Furthermore, the higher the magnitude of the wave-length shift, the greater the O₂ release and the lower the O₂ affinity, as seen with the p₅₀ correlation. Importantly, the bathochromic shift's magnitude in SCD samples significantly correlated to the percentage of HbSS. The dependence of HbSS O₂ affinity on its concentration is consistent with earlier research. From a diagnostic standpoint, this finding proves that our assay may be utilized to measure Hb-O₂ affinity and the percentage of HbSS, which are clinically important in monitoring SCD.

Both HbAA and HbSS variants displayed a reduction in oxygen content with partial deoxygenation, which also manifested as a bathochromic shift in the wavelength of the Soret peak. However, the magnitude of the bathochromic shift was significantly greater for HbSS than for HbAA. There are various possible explanations for this dichotomy. The absorption wave-length of hemoglobin is largely determined by the tertiary and quaternary structures of the protein. Notably, the visible absorption wavelength (350-650 nm) spectra of Hb are determined by the tertiary structure changes which explicitly include hemoglobin environment changes (FIG. 12). In reported experiments, multiple effectors can distort protein conformations. Changing solvent-solute interactions due to increased H⁺ ions in the solution with increased acidity is a potential contributor that may affect both HbAA and HbSS. Increased H⁺ affects the Hb conformation, facilitating O₂ release through the heme-globin interaction. Typically, the bathochromic shift both for HbAA and HbSS can easily be obtained in the acidic condition, at those conditions, the heme ring becomes protonated, which leads to the release of O₂. Furthermore, the localization of sulphite ions to oxygen binding sites may also affect the protein conformation, resulting in the bathochromic shift for HbAA and HbSS. We speculate that the key effector that defines the greater magnitude of bathochromic shift for HbSS is its unique heme environment at lower pH, which otherwise differs from HbAA (FIG. 12). We observed more significant differences in the CD spectra Soret band and aromatic bands of HbSS from those of HbAA in the deoxygenated state at 6.9 pH. Changes in the CD deoxygenated. Soret band have been correlated with the heme-globin interaction environment, which directly reflects the oxygen affinity. HbSS has been reported with slightly different β aromatic residue chains, which may contribute to its distinguished heme environment.

In this method, the purified hemoglobin concentration used is 2.8 mg dl⁻¹ (FIG. 7), more than 10 000 times less than the critical intracellular concentration of ~34 g dl⁻¹ for HbSS polymerization. Therefore, we don't anticipate the polymerization of purified HbSS upon deoxygenation in this assay. We also observed a notably greater bathochromic shift in RBCs for both sickle and normal compared to purified Hb (Table 2) & (FIG. 9). This result could be due to intracellular differences in pH and 2,3-DPG, which are known to affect Hb-O₂ dissociation. A complementary investigation is needed to explain the possible mechanisms contributing to the differences in the heme environment of HbSS at lower pH as it happens physiologically. It is not straightforward to study these variables of pH, solvent, SO₃—O₂ exchange, heme-heme interaction, and polymerization contributions individually because these effectors are correlated. Optical absorption may be a valuable tool for future studies to study these mechanisms. Recent studies report the effects of 2,3-DPG on improving O₂ retention of HbSS but also lowered intracellular pH; thus, the bathochromic shift can also be studied to understand the mechanisms by which 2,3-DPG plays into O₂ retention and polymerization of HbSS.

The full absorption spectrum (350-750 nm) and the four optical variables (bathochromic shift, peak wavelength, AUC, and FWHM) data sets were used to detect HbSS and HbAA variants using the principal component analysis (PCA) algorithm, an unsupervised dimensional reduction tool. Our study high-lights the diagnostic potential of using this method to screen for HbSS and HbAS according to the significant cluster differences between HbSS and HbAA variants that PCA revealed and to stratify SCD patients based on treatment such as transfusion, hydroxyurea and treatment-Naïve SCD patients. We believe that our assay can screen other Hb oxygen affinity abnormalities in addition to HbSS. Robustness and repeatability of the assay support this finding (FIG. 8).

Sodium metabisulfite is a chemically stable antioxidant food additive, which has been used to deoxygenate blood components. Sodium metabisulfite based deoxygenation has been used to induce sickling of RBCs from individuals with SCD. Due to its stability and well-established use in the literature, we used sodium metabisulphite in buffer with a pH of 6.9 to controllably deoxygenate purified hemoglobin, intact RBCs, and whole blood. Sodium metabisulfite dissolves in aqueous buffer to produce sodium bisulfate: Na₂S₂O₅+H₂O→2Na⁺+2HSO⁻. Oxyhemoglobin releases oxygen to result in deoxyhemoglobin and dissolved oxygen: HbO₂→Hb+O₂. Sodium bisulfate is a commonly used reducing agent that readily reacts with dissolved oxygen and is converted into sodium hydrogen sulfate: 2Na⁺+2HSO⁻+O→2NaHSO. Scavenging of dissolved oxygen by sodium bisulfate leads to increased deoxygenation of oxyhemoglobin in the sample. Deoxygenation reaction of sodium metabisulfite is controllable by adjusting its concentration and time, which allows partial deoxygenation of blood components, as we have shown in this study.

The method presented here is designed to assess Hb-O₂ dissociation and eventually determine Hb-O₂ affinity based on the magnitude of Soret band shift but not designed to allow recordings of ODCs over the whole pO₂ range. Future development of this method would include further quantification of the Soret band with the goal to provide absolute quantification of SO₂ and pO₂ for each sample to construct the ODCs. This study also lays the foundation for understanding optical absorption parameters that could differentiate Hb variants with abnormal oxygen affinity and motivates investigation to understand HbSS deoxygenation response dynamics and cellular oxygen availability assessment, which is critical for emerging pharmaceutical and genome editing-based therapies for SCD. In addition, we report evidence that could be used to improve the diagnostic accuracy of systems that use light absorption spectrophotometry, which may be affected by Hb variants and Hb-O₂ affinity abnormalities.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A high throughput assay for screening hemoglobin oxygen affinity modifying agents, the assay comprising:
a well plate configured to receive a plurality of hemoglobin, red blood cells, or blood samples that have been deoxygenated and at least one oxygen affinity modifying agent;
a UV-VIS spectroscopic microplate reader that is configured to determine an optical signature of hemoglobin, red blood cells, or blood in each well; and
a processor configured for comparing the determined optical signature to a control optical signature in each well wherein differences between the determined optical signature and the control optical signature is indicative of the agents' effect on hemoglobin oxygen affinity.

2. The assay of claim 1, wherein the processor is configured to determine differences of absorption spectra of oxygenated and deoxygenated hemoglobin, red blood, and/or blood obtained from the subject by comparing the determined absorption spectra differences to a control value.

3. The assay of claim 1, wherein the processor is configured to determine differences of absorption spectra by comparing a first optical absorption spectrum of oxygenated hemoglobin, red blood cells, or blood with a second optical absorption spectrum of deoxygenated hemoglobin, red blood cells, or blood administered the at least one oxygen affinity modifying agent.

4. The assay of claim 3, wherein the processor is configured to determine differences in at least one of a bathochromic shift and/or hypochromic shift in peak wavelength from the first absorption spectra to the second absorption spectra.

5. The assay of claim 4, wherein the magnitude of bathochromic shift in peak wavelength is indicative of at least one hemoglobin oxygen affinity or rate of hemoglobin deoxygenation.

6. The assay of claim 5, wherein an increase in magnitude of biochromatic shift and/or hypochromic shift in peak wavelength compared to a control is indicative of decreased hemoglobin oxygen affinity or increased hemoglobin deoxidation.

7. The assay of claim 5, wherein processor is configured to determine differences of area under a curve of and/or full width half maximum of peak wavelengths of the first absorption spectra and the second absorption spectra are indicative oxygen affinity modifying effect of the agent.

8. The method of claim 1, wherein the absorption spectra of the oxygenated and chemically deoxygenated hemoglobin are measured at the same pH from about 6.5 to about 7.0, preferably about 6.8 to less than 7.0, or more preferably, about 6.9.

9. The assay of claim 1, wherein the hemoglobin, red blood cells, and/or blood is chemically deoxygenated by mixing the hemoglobin, red blood cells, and/or blood with an amount of chemical deoxygenant effective to deplete oxygen from the hemoglobin.

10. The assay of claim 9, wherein the chemical deoxygenant comprises sodium metabisulfite.

11. A high throughput method for screening oxygen affinity modifying agents, the method comprising:
mixing hemoglobin, red blood cells, and/or whole blood samples with a buffer solution,
deoxygenating the mixed hemoglobin, red blood, and/or blood samples and buffer solution;
adding the mixed hemoglobin, red blood, and/or blood samples and buffer solution to wells of a well plate;
adding at least one oxygen affinity modifying agent from a library of agents to the wells;
determining absorption spectra of deoxygenated hemoglobin, red blood, and/or blood in the wells; and
comparing the determined absorption spectra to a control absorption spectra of oxygenated and/or deoxygenated hemoglobin, red blood, and/or blood mixed with a similar buffer solution, wherein the absorption spectra differences are indicative of the agents effect on modulating hemoglobin oxygen affinity of the hemoglobin, red blood, and/or blood.

12. The method of claim 11, wherein the differences of the absorption spectra include at least one of a bathochromic shift and/or hypochromic shift in peak wavelength from the determined absorption spectrum to the control absorption spectrum.

13. The method of claim 12, wherein the magnitude of bathochromic shift in peak wavelength is indicative of at least one hemoglobin oxygen affinity or rate of hemoglobin deoxygenation.

14. The method of claim 13, wherein an increase in magnitude of bathochromic shifty and/or hypochromic shift in peak wavelength is indicative of decreased hemoglobin oxygen affinity or increased hemoglobin deoxidation.

15. The method of claim 11, wherein the hemoglobin, red blood cells, and/or blood is chemically deoxygenated by mixing the hemoglobin, red blood cells, and/or blood with an amount of chemical deoxygenant effective to deplete oxygen from the hemoglobin.

16. The method of claim 16, wherein the chemical deoxygenant comprises sodium metabisulfite.
